(19) United States
(12) Patent Application Publication
(10) Pub. No.: US 2014/0100585 A1
(43) Pub. Date: Apr. 10, 2014

(54) MEDICAL DEVICE HAVING AN ELECTRO-MAGNETIC DEVICE TIP AND RELATED METHOD OF USE

(71) Applicant: BOSTON SCIENTIFIC SCIMED, INC., Maple Grove, MN (US)

(72) Inventors: James M. Anderson, Fridley, MN (US); Jan Weber, Maastricht (NL); Robert T. Chang, Belmont, CA (US)

(21) Appl. No.: 14/048,309

(22) Filed: Oct. 8, 2013

Related U.S. Application Data
(60) Provisional application No. 61/711,334, filed on Oct. 9, 2012.

(51) Int. Cl. A61B 17/22

(52) U.S. Cl. CPC A61B 17/22012
USPC 606/128; 606/191

(57) ABSTRACT

A medical device may include an elongate shaft having a longitudinal axis and a distal end. The medical device may include a distal tip having at least one electromagnetic coil disposed adjacent the distal end and a plurality of bulbous elements disposed about the longitudinal axis. The plurality of bulbous elements may be operatively associated with the at least one electromagnetic coil such that the plurality of bulbous elements may translate relative to the longitudinal axis in response to activation of the at least one electromagnetic coil. A method of crossing an obstruction may include approaching the obstruction with a medical device, supplying alternating current to at least one electromagnetic coil, and advancing the medical device into engagement with the obstruction.

### TECHNICAL FIELD

This disclosure generally relates to percutaneous medical devices, and more specifically, to percutaneous medical devices designed to navigate tortuous vasculature and to cross obstructions.

### BACKGROUND

Heart disease is a major problem in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. These and other minimally invasive techniques typically involve the use of a catheter, which is advanced over a guidewire such that the balloon is positioned adjacent a stenotic lesion. Where the vasculature to be navigated is particularly torturous or obstructed with lesions, advancing the catheter can be difficult.

During minimally invasive procedures, embolic debris can be separated from the wall of the blood vessel. If this debris enters the circulatory system, it could block other vascular regions including the neural and pulmonary vasculature, both of which are highly undesirable.

### SUMMARY

A medical device may include an elongate shaft having a longitudinal axis and a distal end, at least one electromagnetic coil disposed adjacent the distal end, and a distal tip including a plurality of magnetized bulbous elements disposed about the longitudinal axis, wherein the plurality of magnetized bulbous elements is operatively associated with the at least one electromagnetic coil.

A method of crossing an obstruction in a lumen of a vessel may include inserting a medical device percutaneously into the lumen, the medical device including an elongate shaft having a longitudinal axis and a distal end, at least one electromagnetic coil disposed adjacent the distal end, and a distal tip including a plurality of magnetized bulbous elements disposed about the longitudinal axis, wherein the plurality of magnetized bulbous elements are operatively associated with the at least one electromagnetic coil. The method may further include advancing the medical device toward the obstruction, supplying alternating current to the at least one electromagnetic coil, thereby causing the plurality of magnetized bulbous elements to vibrate, and advancing the medical device into engagement with the obstruction.

Although discussed with specific reference to use within the circulatory vasculature of a patient (i.e., a tortuous artery, for example), medical devices and methods of use in accordance with the disclosure may be adapted and configured for use in other parts of the anatomy, such as the digestive system, the respiratory system, or other parts of the anatomy of a patient.

### BRIEF DESCRIPTION OF THE DRAWINGS

The present disclosure may be more completely understood in consideration of the following detailed description of various embodiments in connection with the accompanying drawings, in which:

FIG. 1 is a schematic partial cross-sectional view of an exemplary tortuous vessel;

FIG. 2 is a schematic partial cross-sectional view of an exemplary stenosed or diseased vessel;

FIG. 3 is a schematic view of an exemplary medical device having a distal tip, in accordance with the present disclosure;

FIG. 4 is a schematic view of a medical device including an exemplary electromagnetic coil;

FIG. 5 is a schematic view of a medical device including an exemplary electromagnetic coil;

FIG. 6A is a schematic partial cross-sectional view of an exemplary electromagnetic coil;

FIG. 6B is a schematic partial cross-sectional view of an exemplary electromagnetic coil;

FIG. 6C is a schematic partial view of an exemplary electromagnetic coil;

FIG. 6D is a schematic partial cross-sectional view of an exemplary electromagnetic coil;

FIG. 7A is a schematic partial side view of a distal tip in a straightened condition;

FIG. 7B is a schematic partial side view of a distal tip in a bent condition;

FIG. 8 illustrates an example distal tip having an electromagnetic coil arrangement such as that shown in FIG. 6D;

FIG. 9 illustrates an example distal tip having an electromagnetic coil arrangement such as that shown in FIG. 6B;

FIGS. 10A-10B illustrate an example distal tip having an electromagnetic coil arrangement such as that shown in FIG. 6C;

FIGS. 11A-11C illustrate alternative lumen placements within an example bulbous element of an example distal tip;

FIG. 12A is a schematic partial cross-sectional view of an example distal tip approaching an obstruction;

FIG. 12B is a schematic partial cross-sectional view of an example tip engaging an obstruction;

FIG. 13 is a schematic partial cross-sectional view of an example tip engaging a lesion or stenosis including an exemplary embolic protection filter; and

FIG. 14 is a schematic partial cross-sectional view of an exemplary medical device traversing the tortuous vessel.

While embodiments of the present disclosure are amenable to various modifications and alternative forms, specifics thereof have been shown by way of example in the drawings and will be described in greater detail below. It should be understood, however, that the intention is not to limit aspects of the disclosure to the particular embodiments described. On the contrary, the intention is to cover all modifications, equivalents, and alternatives falling within the spirit and scope of the present disclosure.

### DETAILED DESCRIPTION

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

For the following defined terms, these definitions shall be applied, unless a different definition is given in the claims or elsewhere in the specification.

The terms “upstream” and “downstream” refer to a position or location relative to the direction of blood flow through a particular element or location, such as a vessel (i.e., the aorta) or vessel lumen, a heart valve (i.e., the aortic valve), and the like.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range, including the endpoints (e.g. 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

In some instances, it may be desirable for a medical device to traverse tortuous, circuitous, or narrow vessels. In addition, the medical device may be required to cross calcified plaques, lesions, or obstructions attached to or embedded in vessel walls. During advancement of the medical device through the vasculature, tight turns may pose navigational problems and narrow operating conditions may pose a danger of dislodging stenotic or embolic material or other debris. The resulting dislodged material may be carried downstream with the blood flow and may cause obstruction of smaller vessels. While the present disclosure may be applicable to catheters or other elongated medical devices of any size (i.e., about 1 French to about 7 French to about 17 French or more), the discussion is particularly applicable to larger bore devices (i.e., greater than about 12 French), where the larger size comes with an accompanying inherent stiffness.

The present disclosure addresses an exemplary medical device having a distal tip including a plurality of bulbous elements. The distal tip may include an electromagnetic coil, designed to energize the bulbous elements, causing them to translate and/or vibrate along or around an elongate shaft. In some embodiments, vibration of the plurality of bulbous elements may facilitate advancing the medical device without causing injury or trauma to the vessel walls. In some embodiments, vibration of the plurality of bulbous elements may facilitate removal of calcified plaque, lesions, or obstructions from a vessel wall, thereby opening or widening the vessel lumen.

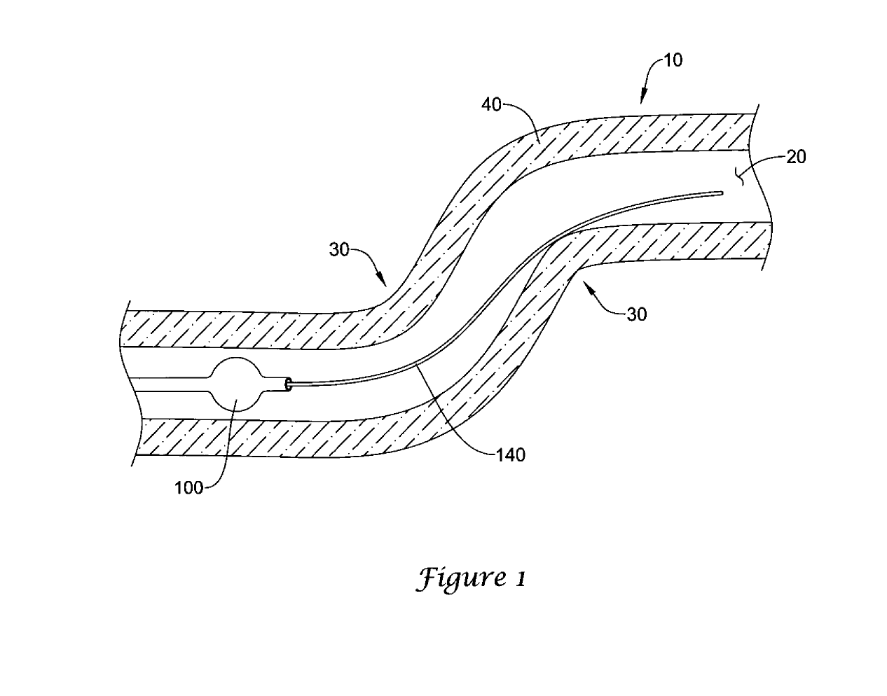

As seen in FIG. 1, a medical device 100 may be required to navigate through a tortuous vessel 10 having a lumen 20 including one or more sharp bend(s) 30. As the medical device 100 navigates through the bend(s) 30, it may scrape against a vessel wall 40, causing injury to the vessel wall 40. In addition, passage through the sharp bend(s) 30 along a guidewire 140 may cause a sharp angle to form between guidewire 140 and the medical device 100. As can be visualized in FIG. 1, the distal portion of the guidewire 140 lies at an angle to a portion of the guidewire 140 disposed proximal to the medical device 100. As the medical device 100 approaches a sharp bend 30, the angle may become more acute, exerting pressure, or a bending load or moment, on a portion of the guidewire 140. That pressure may result in a kink forming in the guidewire 140. A kink may create a sharp edge or corner in the guidewire 140 that may nick the vessel wall 40, thereby causing injury. In addition, delivery of the medical device 100 over the guidewire 140 may transfer enough force to the guidewire 140, through side loading for example, to cause the guidewire 140 to cut or slice through the vessel wall 40 and/or other surrounding tissue(s) around the sharp bend(s) 30. Delivery of the medical device 100 over the guidewire 140 may also cause friction between the medical device 100 and the guidewire 140, resulting in navigational problems for the user and/or may lead to a situation where the medical device 100 cannot be moved any further distally.

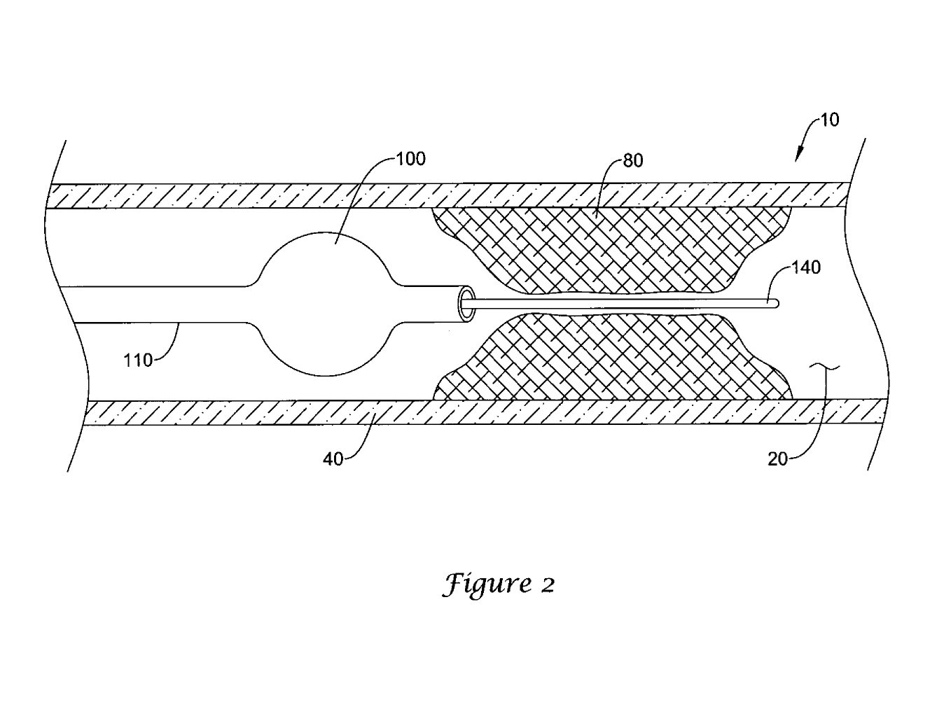

In some treatment procedures, a medical device 100 may be required to traverse a lumen 20 of a vessel 10 that is narrowed or diseased by a calcified plaque, lesion, or obstruction 80, as illustrated in FIG. 2. With or without the aid of a guidewire 140, the medical device 100 may scrape or directly impact a calcified plaque, lesion, or obstruction 80 disposed within the lumen 20 adjacent or attached to the vessel wall 40. That action may cause vulnerable plaque, embolic material or debris, or the like to be released into the bloodstream.

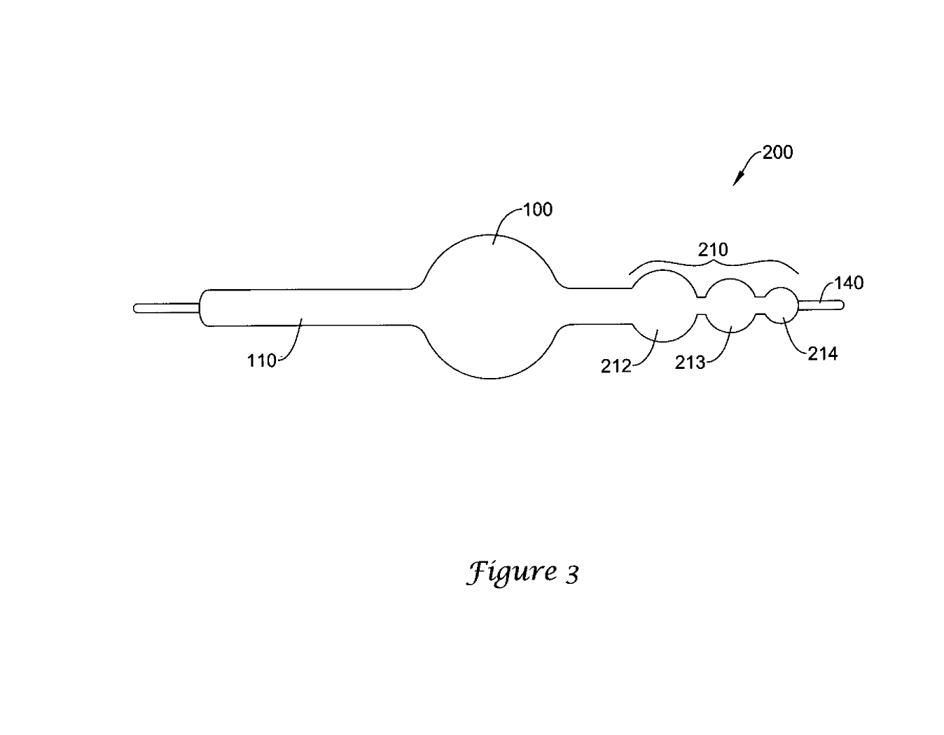

In some embodiments, an example medical device 100 may include a distal tip 200, as illustrated in FIG. 3. An elongate shaft 110 may include or extend through the medical device 100 and the distal tip 200 along a central longitudinal axis. The elongate shaft 110 may be tubular, with a proximal end, a distal end, and a lumen (not shown) extending between the proximal end and the distal end. The distal tip 200 may include a plurality of bulbous elements 210 disposed about the longitudinal axis. In some embodiments, the plurality of bulbous elements 210 may be carried on, around, or over the elongate shaft 110. In some embodiments, the plurality of bulbous elements 210 may include at least three bulbous elements, such as a first bulbous element 212, a second bulbous element 213, and a third bulbous element 214. The plurality of bulbous elements 210 may have diameters or outer extents that decrease respectively in a distal direction, and the plurality of bulbous elements 210 may be mounted on the elongate shaft 110 with space left between adjacent elements. In some embodiments, the plurality of bulbous elements 210 may be integrally formed with the distal tip 200 and the elongate shaft 110. In some embodiments, a guidewire 140 may extend completely through the lumen in the elongate shaft 110.

The elongate shaft 110 may have sufficient performance characteristics (i.e., flexibility, pushability, tensile strength, etc.) to navigate through tight bends and corners of tortuous vasculature, such as, but not limited to the sharp bend(s) 30 of the vessel 10. Suitable example materials for the elongate shaft 110 may include a polymer, a ceramic, a metallic or metallic alloy, a metallic-polymeric composite, combinations thereof (which in some embodiments may include a braid or coil, for example), or the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials, and the like. Persons skilled in the art, however, will appreciate that any suitable material providing desired performance characteristics and biocompatibility may be used, without departing from the scope and spirit of the present disclosure.

In some embodiments, the plurality of bulbous elements 210 may be substantially spherical in shape, although other suitable bulbous shapes including but not limited to, ovoid, elliptical, rounded, bulb-shaped, polygonal, and irregular are also contemplated. In some embodiments, the plurality of bulbous elements 210 may each decrease in size from a proximalmost, first bulbous element 212 distally to a distalmost, third bulbous element 214, such that the proximalmost, first bulbous element 212 is the largest of the plurality of bulbous elements 210 and the distalmost, third bulbous element 214 is the smallest of the plurality of bulbous elements 210. In some embodiments, the plurality of bulbous elements 210 may each have a similar or the same diameter or outer extent, or they may be arranged in some other manner as desired.

In some embodiments, the plurality of bulbous elements 210 may be formed of a relatively rigid and/or radiopaque material. In some embodiments, the plurality of bulbous elements 210 may be formed of or include a metallic material, a metallic alloy, a ceramic material, a rigid or high performance polymer, a metallic-polymer composite, combinations thereof, and the like. Some examples of some suitable materials may include metallic materials and/or alloys such as stainless steel (e.g. 304v stainless steel or 316L stainless steel), nickel-titanium alloy (e.g., nitinol, such as super elastic or linear elastic nitinol), nickel-chromium alloy, nickel-chromium-iron alloy, cobalt alloy, nickel, titanium, platinum, or alternatively, a polymer material, such as a high performance polymer, or other suitable materials, and the like. In some embodiments, the plurality of bulbous elements 210 may be made from a homogenous material or a mixture of materials. In some embodiments, the plurality of bulbous elements 210 may be made by joining distinct areas of different materials, such as by injection molding, adhesive attachment, welding or soldering, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some embodiments, the plurality of bulbous elements 210 may be mixed with, may be doped with, may be coated with, or may otherwise include a radiopaque material. Radiopaque materials are understood to be materials capable of producing a relatively bright image on a fluoroscopy screen or another imaging technique such as X-ray during a medical procedure. This relatively bright image aids the practitioner and/or clinician in determining the location and/or placement of the medical device. Suitable radiopaque materials may include, but are not limited to, bismuth subcarbonate, iodine, gold, platinum, palladium, tantalum, tungsten or tungsten alloy, and the like.

In some embodiments, an outer surface of one or more of the plurality of bulbous elements 210 may be substantially smooth. In some embodiments, an outer surface of each of the plurality of bulbous elements 210 may be substantially smooth. A smooth outer surface on the plurality of bulbous elements 210 may facilitate advancing the medical device 100 within a tortuous vessel 10, around the sharp bend(s) 30, and/or through a soft stenotic deposit or lesion.

In some embodiments, an outer surface of one or more of the plurality of bulbous elements 210 may include or be coated with an abrasive material. In some embodiments, an outer surface of each of the plurality of bulbous elements 210 may include or be coated with an abrasive material. An abrasive coating on the outer surface of the plurality of bulbous elements 210 may facilitate the medical device 100 crossing a plaque deposit or a calcified lesion, while traversing a tortuous vessel 10. In some embodiments, an abrasive coating on the outer surface (or an abrasive outer surface) may permit the distal tip 200 to act as a drill or jackhammer to cut away and/or through the plaque or lesion. Some examples of abrasive materials may include, but are not limited to, diamond dust, metallic powders, or the like.

In some embodiments, the plurality of bulbous elements 210 may each include a lumen extending therethrough. In some embodiments, a lumen extending through the plurality of bulbous elements 210 may include a slick or lubricious coating, allowing the plurality of bulbous elements 210 to more easily translate over the elongate shaft 110 (i.e., slide, rotate, etc.) and/or relative to the longitudinal axis. In some embodiments, the plurality of bulbous elements 210 is slidingly disposed about the elongate shaft 110. In some embodiments, a lumen extending through the plurality of bulbous elements 210 may be non-lubricious. Other potentially useful coatings used in some embodiments may include a hydrophobic or hydrophilic coating, a drug-eluting material, an anti-thrombus coating, or other suitable coating depending on the intended use or application. These and other coating materials may be used in particular applications or embodiments as desired by those of skill in the art.

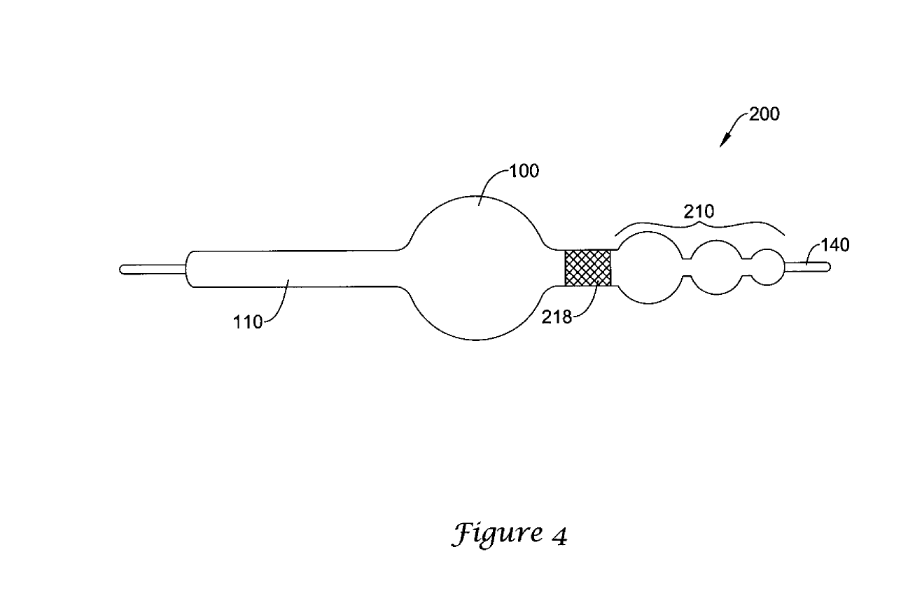

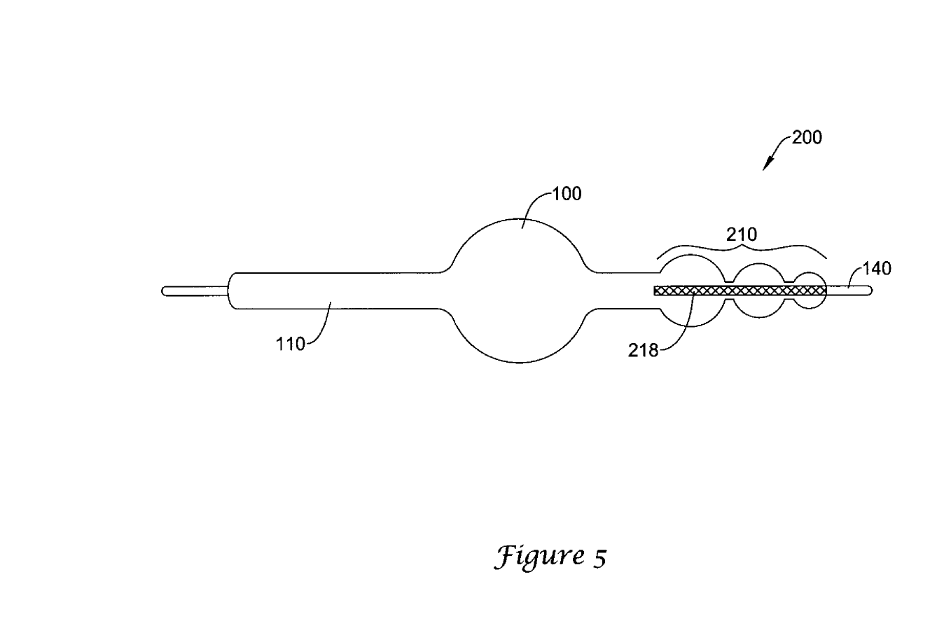

In some embodiments, the distal tip 200 may include at least one electromagnetic coil 218 operatively associated with the plurality of bulbous elements 210, as shown in FIGS. 4 and 5. In some embodiments, one or more of the plurality of bulbous elements 210 may be magnetized, wholly or in part. In some embodiments, magnetization may be accomplished by including a ferromagnetic and/or superparamagnetic material (i.e., iron oxide, dysprosium oxide, gadolinium oxide, neodymium, samarium-cobalt, and the like) mixed or dispersed throughout a non-ferrous substrate or material that forms one or more of the plurality of bulbous elements 210. In some embodiments, the ferromagnetic and/or superparamagnetic material may be magneto-opaque, enabling visualization under magnetic resonance imaging (MRI). Many ferromagnetic and/or superparamagnetic materials are radiopaque and may be materials capable of producing a relatively bright image on a fluoroscopy screen or another imaging technique such as X-ray during a medical procedure. This relatively bright image aids the practitioner and/or clinician in determining the location and/or placement of the medical device. In some embodiments, one or more of the plurality of bulbous elements 210 may be magnetized by including at least one discrete magnetic component 150, as will described below. In some embodiments, polarization (i.e., orientation of the north and south magnetic poles) of the magnetized bulbous elements may be aligned either parallel with or perpendicular to the longitudinal axis, although other suitable or desirable orientations are also possible.

In some embodiments, the plurality of bulbous elements 210 may be configured to translate relative to the longitudinal axis in response to activation of the at least one electromagnetic coil 218. In some embodiments, the plurality of bulbous elements 210 may be configured to translate axially relative to the longitudinal axis in response to activation of the at least one electromagnetic coil 218. In some embodiments, the plurality of bulbous elements 210 may be configured to translate laterally relative to the longitudinal axis in response to activation of the at least one electromagnetic coil 218. In some embodiments, the plurality of bulbous elements 210 may be configured to translate rotationally relative to the longitudinal axis in response to activation of the at least one electromagnetic coil 218. In some embodiments, the plurality of bulbous elements 210 may be configured to translate in at least two of the following directions: axially, laterally, and rotationally, relative to the longitudinal axis in response to activation of the at least one electromagnetic coil 218.

FIG. 4 illustrates at least one electromagnetic coil 218, disposed proximal to the plurality of bulbous elements 210. In some embodiments, the at least one electromagnetic coil 218 may be positioned on an outer surface of the elongate shaft 110, embedded within a wall of the elongate shaft 110, or positioned within a lumen of the elongate shaft 110. One, one or more, or each of the at least one electromagnetic coil 218 may produce an electromagnetic field when energized. Alternating current (AC) may selectively flow through a connector (not shown) to the at least one electromagnetic coil 218, thereby selectively generating one or more electromagnetic fields (not shown). The one or more electromagnetic fields may vary in strength, direction, and/or waveform as driven by the AC source. The one or more electromagnetic fields may exert a varying force on the plurality of bulbous elements 210, resulting in vibration and/or translation of the plurality of the bulbous elements 210 along or about the elongate shaft 110, as will be discussed in more detail below.

It is noted that the reference to alternating current (AC) encompasses all waveforms not being direct current (DC). As such, the term “alternating current” within this description encompasses symmetric as well as asymmetric waveforms with respect to a zero-current axis or reference line. Waveforms may be symmetric (i.e, simple sinus or sine wave shaped, for example) or asymmetric (i.e., sawtooth or saw wave shaped, for example) with respect to rising and declining slopes. Given examples are not intended to be all-inclusive, and one of skill in the art will recognize that additional waveforms are possible. Frequency ranges may extend from 0.01 hertz (Hz) to 1.00 megahertz (MHz), or other suitable frequencies. In some embodiments, a waveform resembling an on-off DC current of a certain timeframe (i.e., 0 to 100 seconds, for example), may be considered a waveform composed of very low frequency AC components.

In some embodiments, a direct current (DC) resistive capacitive (RC) system may be utilized to provide a pulsed surge of DC current. In some embodiments, a DC RC system may have a similar or the same design as an AC system, such as that described above. In a DC RC system, a typical RC control unit may provide the current to the at least one electromagnetic coil 218. A benefit of using an RC system is that capacitive discharge may provide a greater voltage gain of output compared to input (i.e., 5V DC applied to the control unit may produce 50V DC discharged by the capacitor, for example).

The force exerted on the plurality of bulbous elements 210, and the resulting vibration and/or translation of the plurality of the bulbous elements 210, can be exerted in a direction parallel to the longitudinal axis, perpendicular to the longitudinal axis, or at other angles relative to the longitudinal axis. The force exerted may result from the orientation of the magnetic field developed by the at least one electromagnetic coil 218 and the polarization of the plurality of bulbous elements 210. Arranging the at least one electromagnetic coil 218 and the polarization of the plurality of bulbous elements 210 to produce a desired pattern of force, vibration, and/or translation can be accomplished by those of skill in the art. If, for example, it is desired to exert a vibrational force in a direction parallel to the longitudinal axis, vibrations and/or translation may occur axially along the longitudinal axis. Similarly, if, for example, it is desired to exert a vibrational force in a direction perpendicular to the longitudinal axis, vibrations and/or translation may occur laterally and/or the plurality of bulbous elements 210 may rotate about the elongate shaft 110. However, other orientations and/or arrangements may be made, as understood by the skilled artisan. For example, in some embodiments, a particularly useful waveform for advancing forward (i.e., exerting a vibrational force parallel to the longitudinal axis) may be a sawtooth shaped wave having a sharp upward slope and a slower downward slope, which may result in a net forward force on the plurality of bulbous elements 210. In some embodiments, a sawtooth waveform may translate or vibrate the plurality of bulbous elements 210 to produce a back-and-forth axial jackhammer effect.

FIG. 5 illustrates the plurality of bulbous elements 210 disposed about the at least one electromagnetic coil 218. In some embodiments, the at least one electromagnetic coil 218 may be positioned on an outer surface of the elongate shaft 110, embedded within a wall of the elongate shaft 110, or positioned within a lumen of the elongate shaft 110. The at least one electromagnetic coil 218 may produce an electromagnetic field operatively associated with the plurality of bulbous elements 210 when energized. Alternating current (AC) may selectively flow through a connector (not shown) to the at least one electromagnetic coil 218, thereby selectively generating one or more electromagnetic fields (not shown). The one or more electromagnetic fields may vary in strength, direction, and/or waveform as driven by the AC source. The one or more electromagnetic fields may exert a varying force on the plurality of bulbous elements 210, resulting in vibration and/or translation of the plurality of the bulbous elements 210 along or about the elongate shaft 110, as will be discussed in more detail below.

The force exerted on the plurality of bulbous elements 210, and the resulting vibration and/or translation of the plurality of the bulbous elements 210, can be exerted in a direction parallel to the longitudinal axis, perpendicular to the longitudinal axis, or at other angles relative to the longitudinal axis. The force results from the orientation of the magnetic field developed by the at least one electromagnetic coil 218 and the polarization of the plurality of bulbous elements 210. Arranging the at least one electromagnetic coil 218 and the polarization of the plurality of bulbous elements 210 to produce a desired pattern of force, vibration, and/or translation can be accomplished by those of skill in the art. If, for example, it is desired to exert a vibrational force in a direction parallel to the longitudinal axis, vibrations and/or translation may occur axially along the longitudinal axis. Similarly, if, for example, it is desired to exert a vibrational force in a direction perpendicular to the longitudinal axis, vibrations and/or translation may occur laterally and/or the plurality of bulbous elements 210 may rotate about the elongate shaft 110. However, other orientations and/or arrangements may be made, as understood by the skilled artisan. For example, in some embodiments, it may be particularly useful to monitor the current and/or voltage supplied to the at least one electromagnetic coil 218, thereby permitting modification of the waveform (i.e., changing the frequency of a simple sinusoidal shape, for example) to match the resonance frequency of the system. In use, electrical energy supplied to the at least one electromagnetic coil 218 is transferred into vibrational energy of the plurality of bulbous elements 210, and this transfer is more efficient at and/or close to the resonant frequencies of the system. As such, in some embodiments, a feedback system monitoring the electrical signal may be useful to continuously optimize the system's behavior during operation. As the resonant frequency is a complex function of both the electronic as well as mechanical situation of the system, it is expected that a slight but small drift in resonant frequency may occur during operation. Sweeping the waveform frequency around the resonant point may allow for finding and adjusting for drift(s).

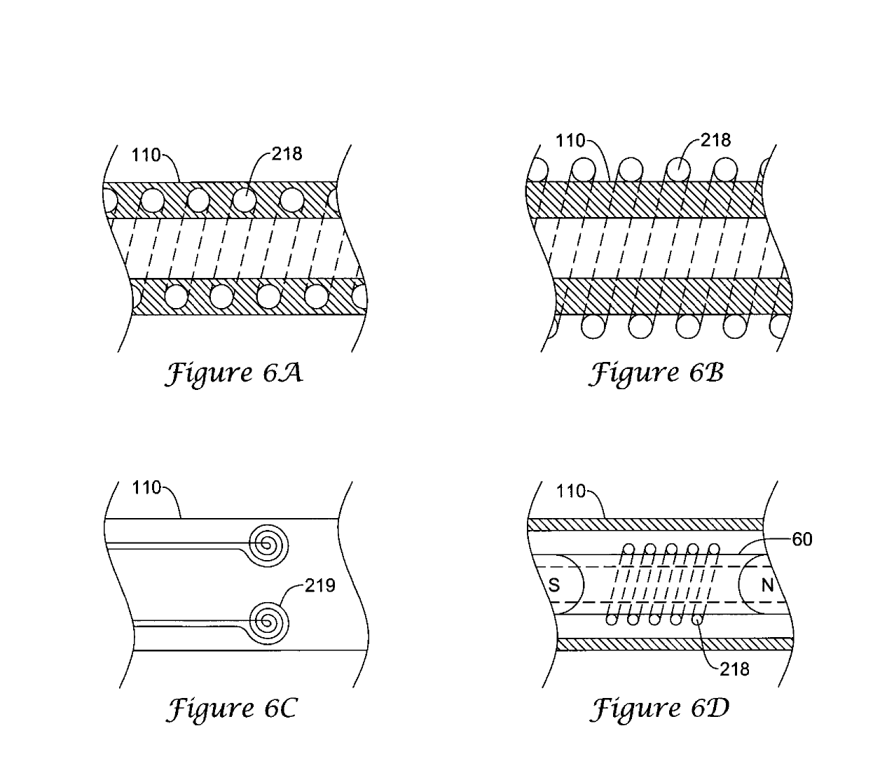

In some embodiments, such as seen in FIG. 6A, the at least one electromagnetic coil 218 may be embedded in a wall of the elongate shaft 110. Those of skill in the art will recognize a number of ways to accomplish such a structure. For example, the at least one electromagnetic coil 218 could be formed on a die or mandrel, and the elongate shaft 110 could be formed around the at least one electromagnetic coil 218. In some embodiments, the at least one electromagnetic coil 218 structure shown in FIG. 6A may be used, for example, in the arrangement shown in FIG. 4. In some embodiments, the at least one electromagnetic coil 218 structure shown in FIG. 6A may be used, for example, in the arrangement shown in FIG. 5.

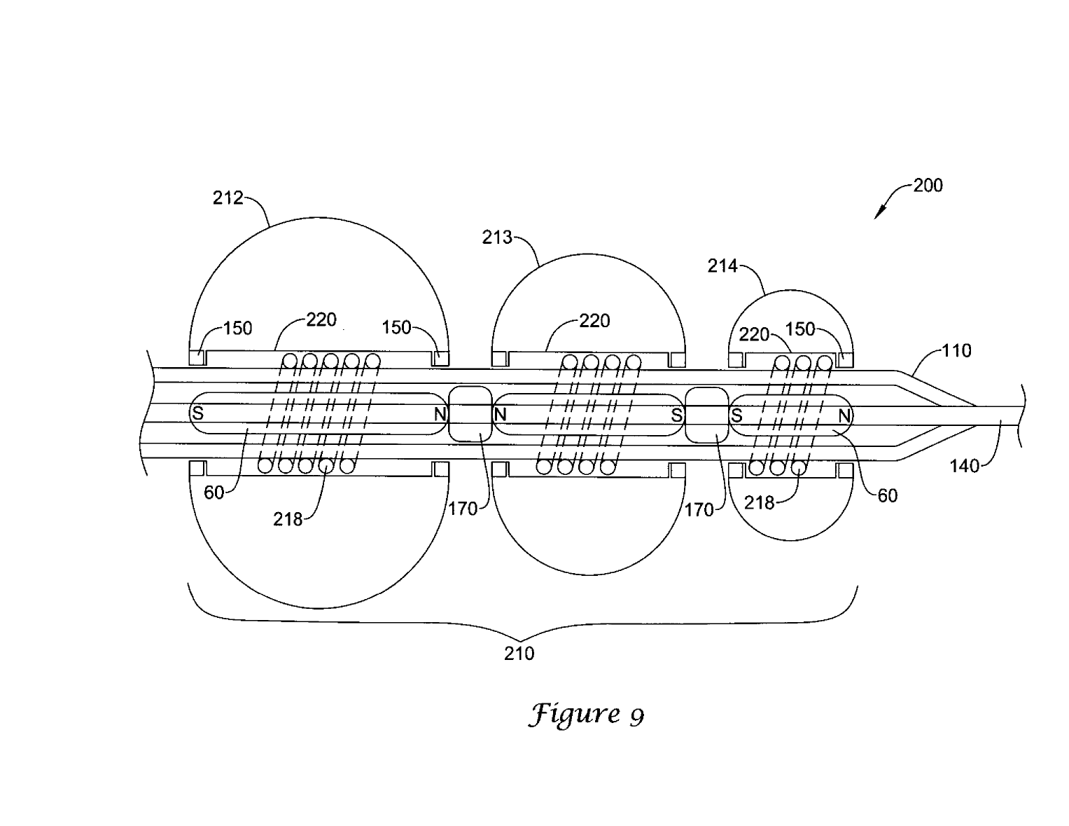

In some embodiments, such as seen in FIG. 6B, the at least one electromagnetic coil 218 may be disposed about, around, or on an outer surface of elongate shaft 110. Those of skill in the art will recognize a number of ways to produce such a structure. For example, the elongate shaft 110 could be formed on a die or mandrel, extruded, or otherwise manufactured, and the at least one electromagnetic coil 218 could be formed, wrapped, or otherwise disposed about or on an exterior surface of the elongate shaft 110. In some embodiments, the at least one electromagnetic coil 218 surrounds the elongate shaft 110. In some embodiments, the at least one electromagnetic coil 218 structure shown in FIG. 6B may be used, for example, in the arrangement shown in FIG. 4. In some embodiments, the at least one electromagnetic coil 218 structure shown in FIG. 6B may be used, for example, in the arrangement shown in FIG. 5. An additional example may be seen in FIG. 9.

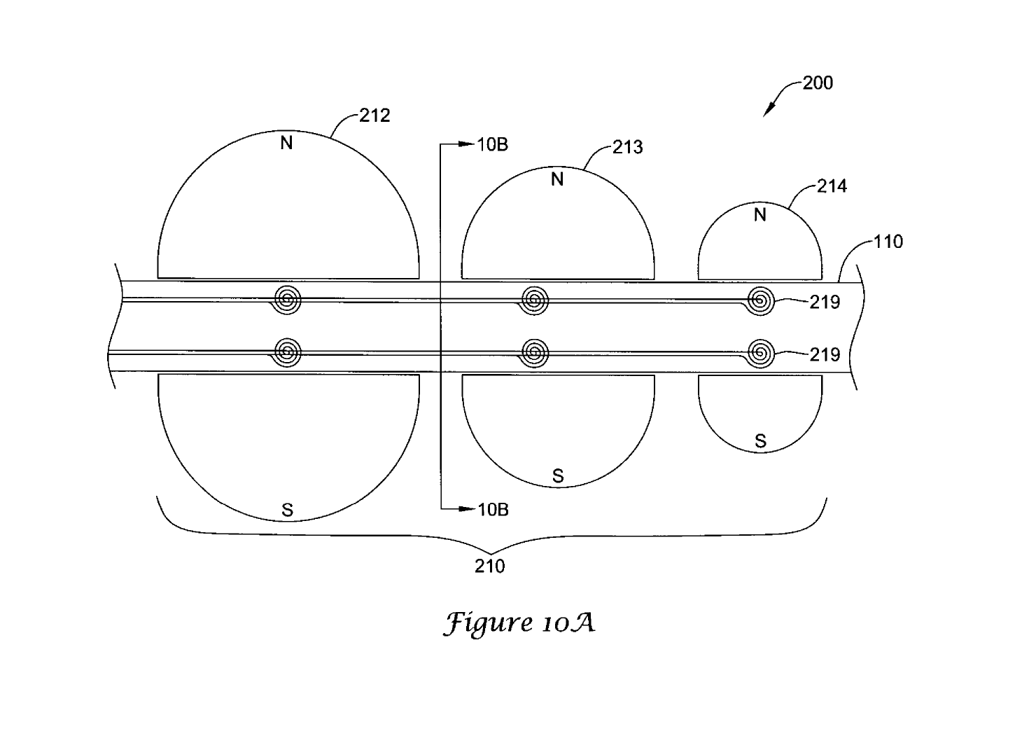

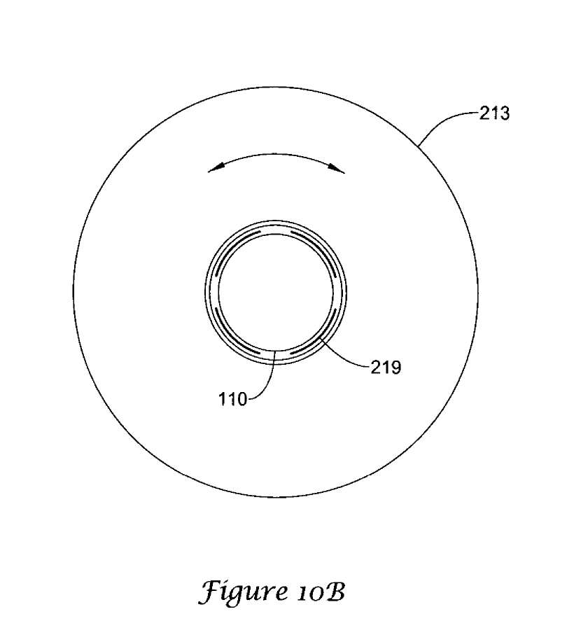

In FIG. 6C, the at least one electromagnetic coil 218 may include a plurality of microcoils 219. The plurality of microcoils 219 may represent a group of thin wires having minimum thickness, wrapped around the elongate shaft 110. In some embodiments, the distal tip 200 may include a plurality of microcoils 219 spaced apart on and/or about an exterior surface of the elongate shaft 110, an arrangement that would allow alternating current to be supplied to selectively activate the plurality of microcoils 219 in a sequential order. In one example, an outer diameter of one microcoil 219 may have dimensions of, but not be limited to, 0.5 mm×0.5 mm. In some embodiments, the plurality of microcoils 219 may be affixed to the exterior surface of the elongate shaft 110, such as by glue or adhesive, or other suitable means. In some embodiments, the plurality of microcoils 219 may be arranged proximally of a distalmost end of the distal tip 200 and may be positioned at various radial angles around the elongate shaft 110. In some embodiments, the at least one electromagnetic coil 218 structure shown in FIG. 6C may be used, for example, in the arrangement shown in FIG. 4. In some embodiments, the at least one electromagnetic coil 218 structure shown in FIG. 6C may be used, for example, in the arrangement shown in FIG. 5. An additional example may be seen in FIGS. 10A-10B.

Referring to FIG. 6D, one, one or more, or each of the at least one electromagnetic coil 218 may include a magnet 60 disposed within the at least one electromagnetic coil 218. In some embodiments, the magnet 60 may be disposed within a lumen of the elongate shaft 110. In some embodiments, the at least one electromagnetic coil 218 may be wrapped around the magnet 60. In some embodiments, the at least one electromagnetic coil 218 may be wrapped around the magnet 60 within the elongate shaft 110. In some embodiments, the at least one electromagnetic coil 218 may be disposed within a wall of the elongate shaft 110. In some embodiments, the at least one electromagnetic coil 218 may be disposed about and/or on an exterior surface of the elongate shaft 110. In some embodiments, the magnet 60 may be a tubular magnet (not shown) disposed about or on an exterior surface of the elongate shaft 110, with the at least one electromagnetic coil 218 wrapped around or on the tubular magnet. In some embodiments utilizing a plurality of magnets 60, the plurality of magnets 60 may be disposed with similar magnetic poles facing towards adjacent magnets 60. That is, in an example embodiment including a first magnet 60 having a north magnetic pole at a distal end thereof, and a second magnet 60 disposed distal of the first magnet 60, a north magnetic pole of the second magnet 60 may be disposed at a proximal end adjacent to the north magnetic pole at the distal end of the first magnet 60. The adjacent similar magnetic poles may be spaced apart by a spacer element 170 disposed between the first and second magnets 60. The spacer element 170 will be described in more detail below. In some embodiments, the at least one electromagnetic coil 218 structure shown in FIG. 6D may be used, for example, in the arrangement shown in FIG. 4. In some embodiments, the at least one electromagnetic coil 218 structure shown in FIG. 6D may be used, for example, in the arrangement shown in FIG. 5. Additional examples may be seen in FIGS. 8-9.

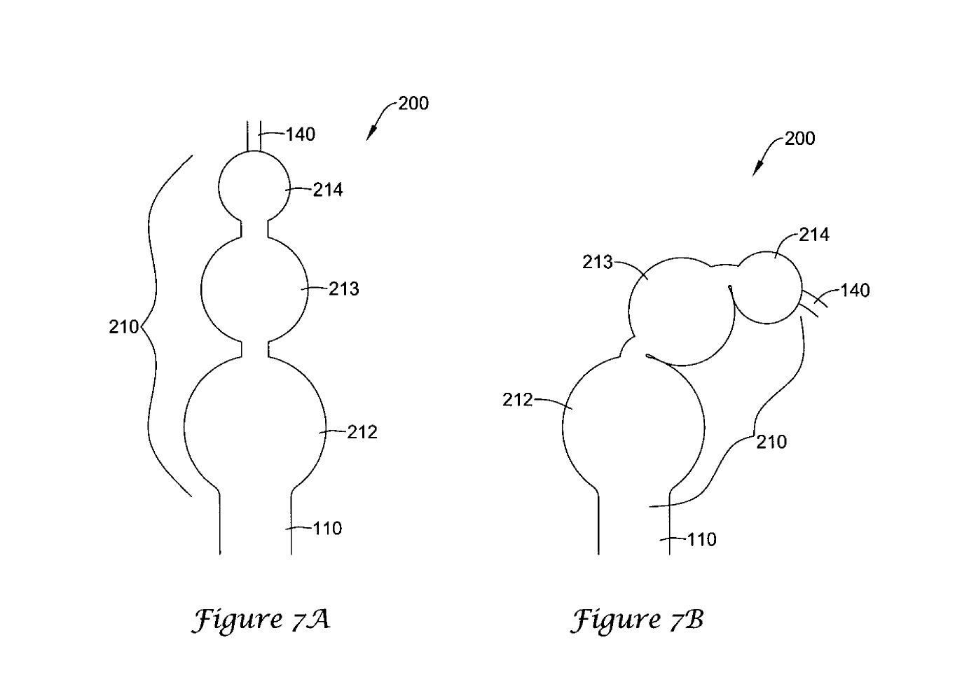

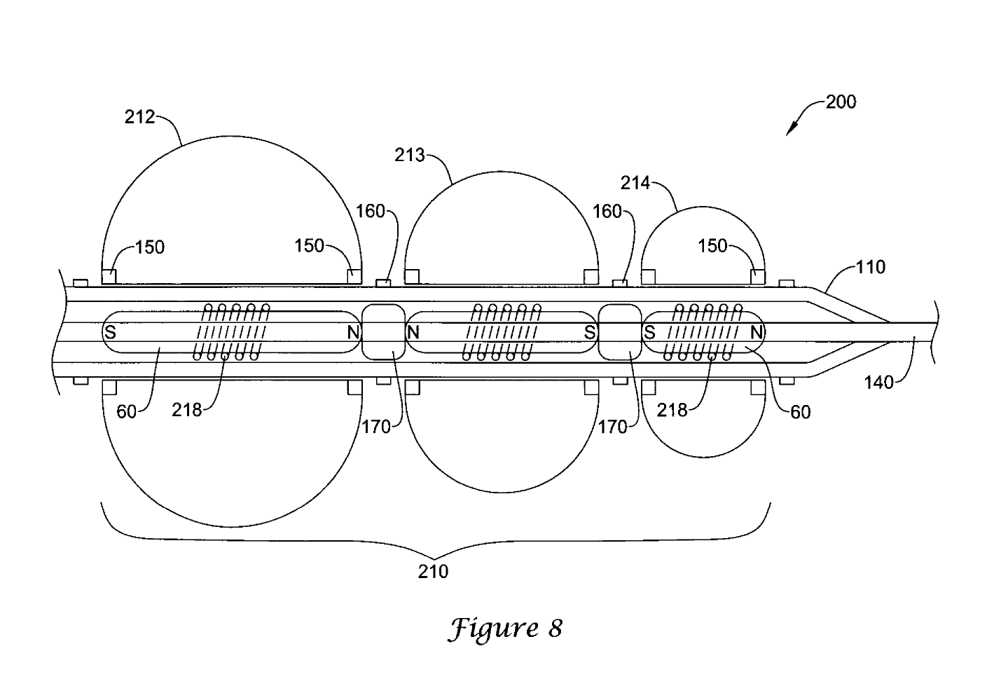

FIGS. 7A-B illustrate an example distal tip 200 configured to selectively actuate from a straightened condition to a bent condition. In some embodiments, the plurality of bulbous elements 210 may be configured to translate laterally relative to the longitudinal axis in response to activation of the at least one electromagnetic coil 218. In some embodiments, the distal tip 200 may be configured to be directionally steerable using selective activation of the at least one electromagnetic coil 218. In some embodiments, alternating current may be supplied to the at least one electromagnetic coil 218. An electromagnetic field may be produced which interacts with the plurality of bulbous elements 210 to cause the plurality of bulbous elements 210 to translate relative to the longitudinal axis. In other words, selective activation of the at least one electromagnetic coil 218 may cause the plurality of bulbous elements 210 to translate laterally relative to the longitudinal axis (i.e., actuate from the straightened condition to the bent condition), thereby permitting the distal tip 200 to be steerable toward a desired direction to facilitate advancement through tortuous vasculature.

As can be seen from FIG. 7B, the plurality of bulbous elements 210 may cooperate such that at a predetermined bending angle, adjacent bulbous elements may come into contact with each other and thus prevent further bending of the distal tip 200 between those adjacent bulbous elements that are in contact with each other. Construction of the distal tip 200 in this manner may distribute the bending along a longer portion of the distal tip 200 in a predetermined curve or radius, so as to avoid forming a sharp angle between a guidewire 140 and a medical device 100 disposed thereon, thereby preventing a kink from forming in the guidewire 140 which may damage or cause injury to a vessel wall 40.

FIG. 8 is a schematic partial cross-sectional view of an example distal tip 200 including the at least one electromagnetic coil 218 encompassing a magnet 60, such as that shown in FIG. 6D. FIG. 9 is a schematic partial cross-sectional view of an example distal tip 200 including the at least one electromagnetic coil 218 encompassing a magnet 60, such as that shown in FIG. 6B. However, these illustrations represent only selected examples, and other combinations using different arrangements as described herein, such as those shown in FIGS. 6A and 6C, are also contemplated.

In some embodiments, the plurality of bulbous elements 210 may each encompass or surround at least one electromagnetic coil 218 and at least one magnet 60. Accordingly, in some embodiments, the at least one electromagnetic coil 218 may include a first electromagnetic coil, a second electromagnetic coil, and a third electromagnetic coil. In some embodiments, the plurality of bulbous elements 210 may include a first bulbous element 212 operatively associated with the first electromagnetic coil, a second bulbous element 213 operatively associated with the second electromagnetic coil, and a third bulbous element 214 operatively associated with the third electromagnetic coil. In some embodiments, each electromagnetic coil/magnet combination may be disposed within the elongate shaft 110 aligned within its respective bulbous element 212, 213, 214. In some embodiments, the at least one electromagnetic coil 218 of each coil/magnet combination may be disposed about or on an exterior surface of the elongate shaft 110 and the magnet 60 is disposed within the elongate shaft 110, while the combination remains aligned within its respective bulbous element 212, 213, 214.

In some embodiments, a spacer element 170 may be disposed between adjacent magnets 60 within the elongate shaft 110. In some embodiments, the spacer element(s) may be disposed within the lumen of the elongate shaft 110. In some embodiments, the spacer element 170 may be configured to and/or sized to space apart adjacent magnets 60. In some embodiments, the spacer element(s) 170 may be formed from or include a radiopaque material.

In some embodiments, the distal tip 200 may include one or more rings or bands 160 disposed about or on an exterior surface of the elongate shaft 110. In some embodiments, the band(s) 160 may be fixedly attached to, or integrally formed with, the exterior surface of the elongate shaft 110 to serve as a mechanical stop limiting axial translation of one or more of the plurality of bulbous elements 210. In some embodiments, the band(s) 160 may be formed from or include a radiopaque material. Generally, the band(s) 160 may be void of ferrous materials and/or non-magnetic in nature. However, magnetic and/or electromagnetic band(s) 160 are contemplated as an alternative means of inciting vibration and/or translation of the plurality of bulbous elements 210.

In some embodiments, one, one or more, or each of the plurality of bulbous elements 210 may include at least one discrete magnetic component 150 disposed therein. In some embodiments, the at least one discrete magnetic component 150 may be embedded in or attached to an outer surface of one of the plurality of bulbous elements 210. The at least one discrete magnetic component 150 may be of any convenient shape, including but not limited to circular or ring-like, rectangular, regular, or irregular. In some embodiments, the at least one discrete magnetic component 150 may be a ring or band disposed about the elongate shaft 110 and/or the longitudinal axis. The at least one discrete magnetic component 150 may be a specialized magnetic device, such as an alnico magnet for example, or a simple piece of ferromagnetic material. Alternatively, the at least one magnetic component 150 may be made up of a complex material including a magnetic substance, such as ferromagnetic powder. The at least one magnetic component 150 may include a magnetic material either partially or wholly.

In some embodiments, the at least one magnetic component 150 may cooperate with the at least one electromagnetic coil 218 to effect axial, lateral, or rotational translation of one, one or more, or each of the plurality of bulbous elements 210 relative to the longitudinal axis and/or the elongate shaft 110. In some embodiments, the at least one magnetic component 150 may be attracted to, repelled by, or otherwise affected by an electromagnetic field produced by activation and/or energizing of the at least one electromagnetic coil 218. In practice, interaction of the at least one magnetic component 150 with the electromagnetic field results in the plurality of bulbous elements 210 being translated along the elongate shaft 110 in a vibratory manner—back and forth in an axial direction, a lateral direction, and/or rotationally about the longitudinal axis and/or the elongate shaft 110.

In some embodiments, including the example illustrated in FIG. 9, one, one or more, or each of the plurality of bulbous elements 210 may include a notch 220 formed within an interior of or as a part of a lumen extending through the bulbous element. In some embodiments, the notch 220 may be disposed about the at least one electromagnetic coil 218, or the at least one electromagnetic coil 218 may be disposed within the notch 220, such that a proximal end of the notch 220 and a distal end of the notch 220 each form a mechanical stop limiting axial translation of the bulbous element about the at least one electromagnetic coil 218 and/or the longitudinal axis. In some embodiments, the at least one discrete magnetic component 150 may be included in and/or form the proximal end and/or the distal end of the notch 220. In embodiments having a notch 220 disposed about at least one electromagnetic coil 218 disposed about or on an exterior surface of the elongate shaft 110, the distal tip 200 may or may not include the band(s) 160 described above, since the notch 220 may cooperate with the at least one electromagnetic coil 218 to form a mechanical stop limiting axial translation of the bulbous element.

Vibration of the plurality of bulbous elements 210 provides a useful means for crossing a plaque, a lesion, or an obstruction 80 in a lumen 20 of the vessel 10. A method of crossing an obstruction in a lumen of a vessel may include inserting a medical device 100, including a distal tip 200 as described herein, percutaneously into the lumen 20 and then advancing the medical device 100 toward the obstruction 80. When the medical device 100 reaches the obstruction 80, alternating current may be supplied to the at least one electromagnetic coil 218, thereby energizing the at least one electromagnetic coil 218 and causing the plurality of bulbous elements 210 to vibrate. As noted above, the operative association of the at least one electromagnetic coil 218 and the plurality of bulbous elements 210 (via magnetization of the bulbous elements or inclusion of the at least one magnetic component 150) produces a back and forth vibratory motion of the plurality of bulbous elements 210. In some embodiments, the plurality of bulbous elements 210 may be carried on or be disposed about the elongate shaft 110 so that they may axially slide back and forth on the elongate shaft 110. The vibratory motion of the plurality of bulbous elements 210 may facilitate advancement of the distal tip 200 and/or the medical device 100 through the obstruction 80.

In some embodiments, the at least one electromagnetic coil 218, in a non-energized state, may be used as a pick-up or sensing element to detect the precise position of the obstruction 80. If forward movement of the elongate shaft 110 causes the plurality of bulbous elements 210 to encounter the obstruction 80, the plurality of bulbous elements 210 may move relative to the at least one electromagnetic coil 218. Movement of the plurality of bulbous elements 210 relative to the at least one electromagnetic coil 218 may generate a current in the at least one electromagnetic coil 218 which can be detected by electronic means.

Additionally, in some embodiments having first, second, and third electromagnetic coils (such as described above, for example), it may be possible to energize only the first electromagnetic coil and the third electromagnetic coil while the second electromagnetic coil, disposed between the first electromagnetic coil and the third electromagnetic coil, remains in a non-energized state acting as a sensor coil (i.e., a linear voltage displacement transducer). In some embodiments, the second electromagnetic coil may detect feedback of inductive electromagnetic voltage produced as the corresponding second bulbous element moves relative to the elongate shaft. In operation, some embodiments may energize the first electromagnetic coil and the third electromagnetic coil, resulting in corresponding vibration of the first bulbous element and the third bulbous element. The second bulbous element, disposed between the first bulbous element and the third bulbous element, may be involuntarily translated axially by the first bulbous element and the third bulbous element. In some embodiments, the second bulbous element may be a reactive element, acting as an opposing spring force between the first bulbous element and the third bulbous element (i.e., a second bulbous element formed from rubber or other spring-like material having a reactive or opposing spring force when under compression).

FIGS. 10A-10B illustrate an example distal tip 200 having at least one electromagnetic coil 218 including a plurality of microcoils 219, such as is shown in FIG. 6C. In some embodiments, the plurality of microcoils 219 may be selectively activated as described above, in order to effect sequentially-produced electromagnetic fields. In some embodiments, the plurality of bulbous elements 210 may each be magnetized or include one or more discrete magnetic components 150, such that the sequentially-produced electromagnetic fields cause the plurality of bulbous elements 210 to rotate about the longitudinal axis and/or the elongate shaft 110. In some embodiments, each of the plurality of bulbous elements 210 may be configured to rotate about the longitudinal axis in response to sequential activation of the at least one electromagnetic coil 218 associated therewith.

FIG. 10B illustrates a cross-sectional view of a portion of the example distal tip 200 of FIG. 10A having a second bulbous element 213 disposed about a plurality of microcoils 219 disposed on an exterior surface of the elongate shaft 110. While not explicitly shown in FIG. 10A, the plurality of bulbous elements 210 may have one or more bands 160 disposed between adjacent bulbous elements. The band(s) 160 may function as mechanical stops limiting axial translation of the plurality of bulbous elements 210 and maintaining alignment of the plurality of bulbous elements 210 with the at least one electromagnetic coil 218, as marker bands for visualization of the distal tip 200 during an interventional procedure, and/or as spacers separating adjacent bulbous elements.

During operation of the plurality of microcoils 219, alternating current may be supplied to a first microcoil. Next, alternating current may be discontinued to the first microcoil and instead supplied to a second, adjacent microcoil. Continuing on, alternating current may be selectively supplied, in sequence, to each of the plurality of microcoils 219, thereby generating a moving electromagnetic field. In some embodiments, alternating current may be decreased in the first microcoil while alternating currently is increasingly supplied to the second microcoil. That is, the supply of alternating current does not need to be an on-off switching operation from one microcoil to the next, but may instead be a gradual transition. The moving electromagnetic field cooperates with the associated bulbous element to cause the bulbous element to rotate about the longitudinal axis and/or the elongate shaft 110.

While not explicitly illustrated, one of ordinary skill in the art will recognize that rotational translation of the plurality of bulbous elements 210 is not limited to the configuration shown in FIGS. 10A-10B, or the presence of the plurality of microcoils 219. Various combinations of elements disclosed herein may be arranged in a manner suitable to produce rotational translation of the plurality of bulbous elements 210.

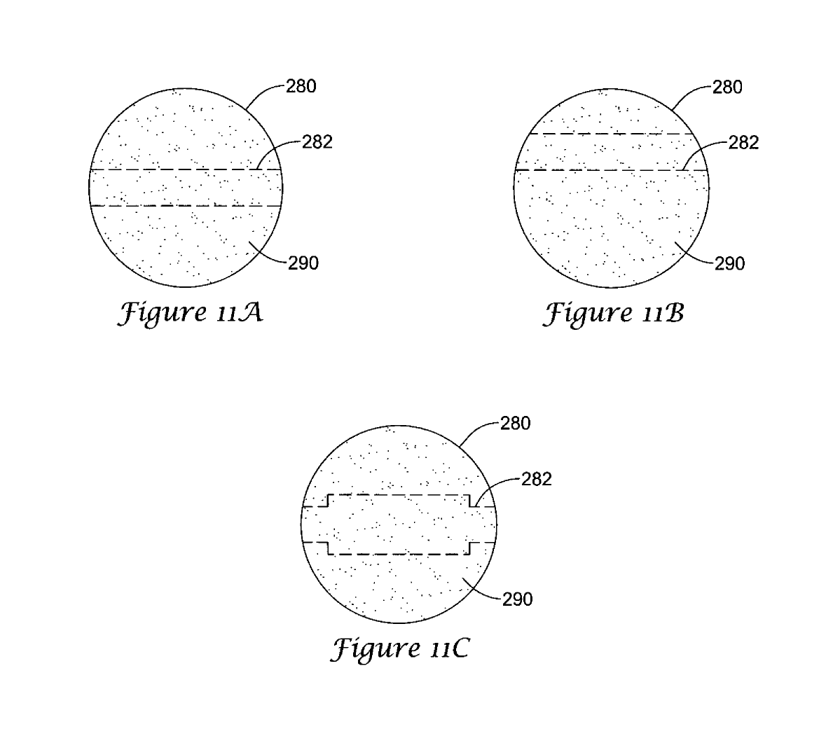

FIG. 11A illustrates an example configuration of one bulbous element 280 of the plurality of bulbous elements 210, wherein a lumen 282 extends through a bulbous element concentric with the longitudinal axis and/or the elongate shaft 110. Bulbous elements having a concentric lumen 282 extending therethrough may translate axially and/or rotationally about the longitudinal axis in a uniform manner. In some embodiments, the plurality of bulbous elements 210 may include a substantially smooth exterior surface, suitable for advancing and/or guiding a medical device 100 through tortuous vasculature. Vibration of smooth bulbous elements having a concentric lumen 282 may facilitate advancement through the vessel 10 and/or through the sharp bend(s) 30. In some embodiments, the plurality of bulbous elements 210 may include an abrasive or abrasive-coated exterior surface, suitable for loosening and/or removing calcified plaque, lesions, or other obstructions within a vessel lumen. Vibration of abrasive bulbous elements having a concentric lumen 282 may produce a back-and-forth, axial jackhammer effect upon the calcified plaque or obstruction. Alternatively, vibration of abrasive bulbous elements having a concentric lumen 282 may produce a rotational drilling effect upon the calcified plaque or obstruction.

FIG. 11B illustrates an example configuration of one bulbous element 280 of the plurality of bulbous elements 210, wherein a lumen 282 extends through a bulbous element offset from a central axis of the bulbous element. Bulbous elements having an offset lumen 282 extending therethrough may translate axially and/or rotationally, but when translating rotationally, translate in an oblong or non-uniform manner about the longitudinal axis and/or the elongate shaft 110. In some embodiments, the plurality of bulbous elements 210 may include a substantially smooth exterior surface, suitable for advancing and/or guiding a medical device 100 through tortuous vasculature. Vibration of smooth bulbous elements having an offset lumen 282 may facilitate advancement through the vessel 10 and/or through the sharp bend(s) 30. In some embodiments, the plurality of bulbous elements 210 may include an abrasive or abrasive-coated exterior surface, suitable for loosening and/or removing calcified plaque, lesions, or other obstructions within a vessel lumen. Vibration of abrasive bulbous elements having an offset lumen 282 may produce a back-and-forth, axial jackhammer effect upon the calcified plaque or obstruction. Alternatively, vibration of abrasive bulbous elements having an offset lumen 282 may produce a rotational drilling effect upon the calcified plaque or obstruction, where the oblong or non-uniform manner of rotation about the longitudinal axis and/or the elongate shaft 110 creates a larger opening (as measured radially relative to an axis of the vessel lumen) through the calcified plaque or obstruction than bulbous elements having a concentric lumen 282.

FIG. 11C illustrates an example configuration of one bulbous element 280 of the plurality of bulbous elements 210, wherein a lumen 282 extends through a bulbous element concentric with the longitudinal axis and/or the elongate shaft 110, wherein the lumen 282 includes a notch integrally formed therewith. The configuration of FIG. 11C may function similar to that of FIG. 11A, the notch being provided as an alternative mechanical stop or means of effecting translation of the plurality of bulbous elements 210, as would be understood by the skilled artisan.

While not explicitly required, the one bulbous element 280 of the plurality of bulbous elements 210 shown in FIGS. 11A-11C includes an optional abrasive material or coating 290, as discussed herein, for illustrative purposes. As noted above, in some embodiments the plurality of bulbous elements 210 may include a substantially smooth exterior surface.

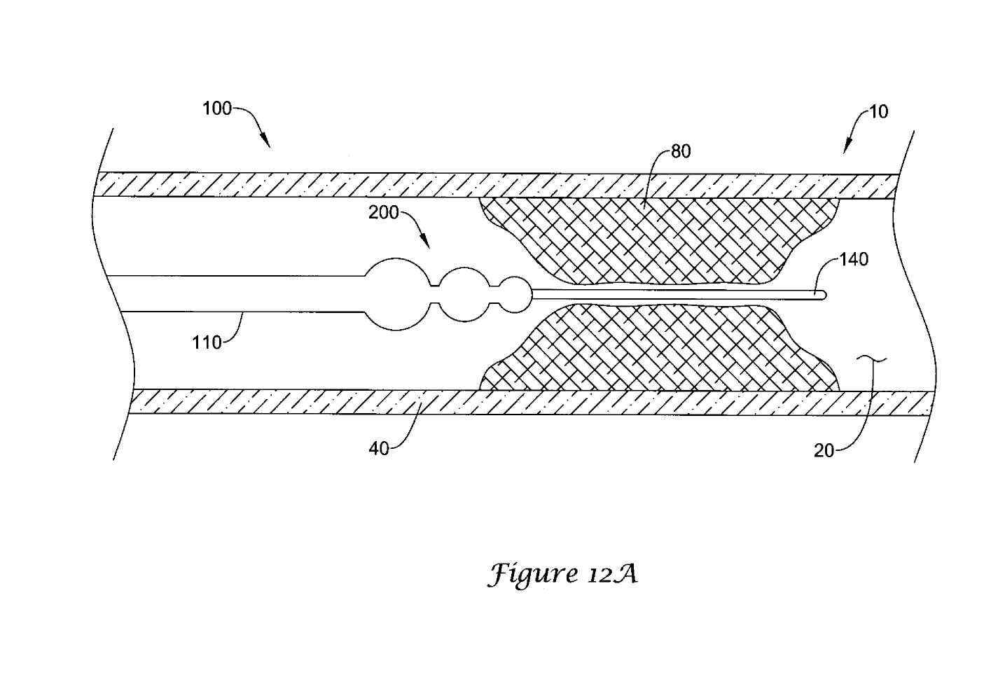

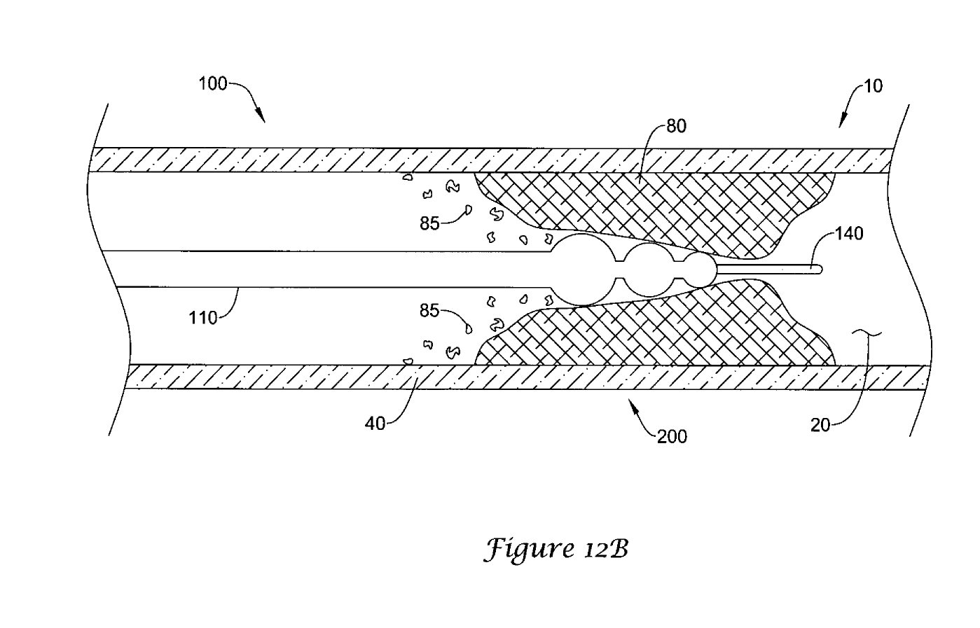

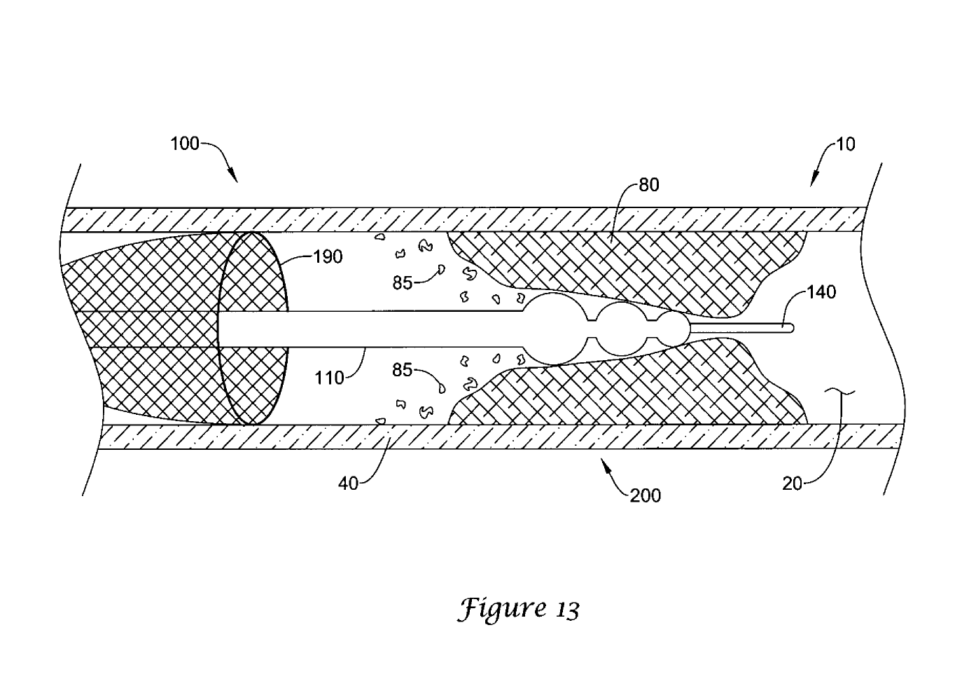

FIGS. 12A-12B are schematic partial cross-sectional views of a medical device 100 disposed within a lumen 20 of a vessel 10 having a calcified plaque, lesion, or obstruction 80 therein. As previously discussed, due to a calcified plaque, lesion, or obstruction 80 attached to or on the vessel wall 40, the lumen 20 of the vessel 10 may become narrowed. A method of crossing an obstruction 80 in a lumen 20 of a vessel 10 may include inserting a medical device 100, in accordance with the disclosure herein, percutaneously into the lumen 20, and advancing the medical device 100 toward the obstruction 80, as shown in FIG. 12A. The method may further include supplying alternating current to the at least one electromagnetic coil 218, thereby causing the plurality of bulbous elements 210 to vibrate. Next, the medical device 100 may be advanced into engagement with the obstruction 80. In some embodiments, advancing the medical device 100 into engagement with the obstruction 80 may result in breaking or release of embolic material 85 from the obstruction 80, as shown in FIG. 12B. In some embodiments, a medical device 100 may include an embolic protection filter 190 coupled to the elongate shaft 110 and disposed proximal of the distal tip 200, as seen in FIG. 13. The method of crossing an obstruction 80 in a lumen 20 of a vessel 10 may further include the step of deploying the embolic protection filter 190 prior to advancing the medical device 100 into engagement with the obstruction 80. In some embodiments, the embolic material 85 may be collected in the embolic protection filter 190. As apparent to those persons skilled in the art, the embolic protection filter 190 may not necessarily be directly attached to the elongate shaft 110.

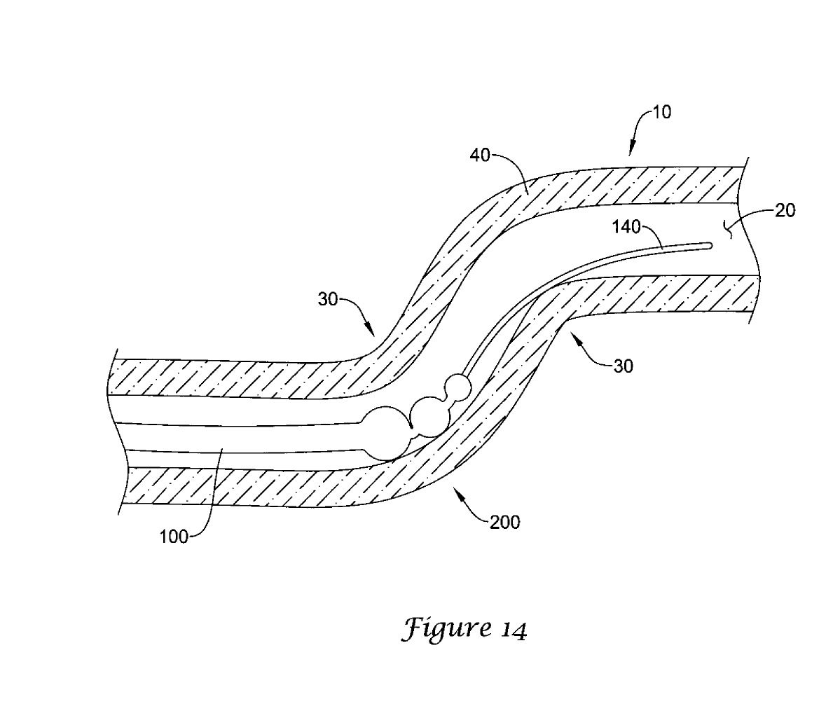

FIG. 14 represents a generic view of navigation of the medical device 100 through the sharp bend(s) 30 of the tortuous vessel 10. As discussed above with respect to FIGS. 7A-7B, the distal tip 200 may be actuated from a straightened condition to a bent condition by selectively supplying alternating current to the at least one electromagnetic coil 218. FIG. 14 illustrates the distal tip 200 in a bent condition safely traversing the sharp bend(s) 30 in accordance with the disclosure herein.

It should be understood that although the above discussion was focused on a medical device and methods of use within the vascular system of a patient, other embodiments of medical devices or methods in accordance with the disclosure may be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the apparatus and/or medical devices described herein with respect to percutaneous deployment may be used in other types of surgical procedures as appropriate. For example, in some embodiments, the medical devices may be deployed in a non-percutaneous procedure, such as an open-heart procedure. Devices and methods in accordance with the invention can also be adapted and configured for other uses within the anatomy.

It should be understood that this disclosure is, in many respects, only illustrative. Changes may be made in details, particularly in matters of shape, size, and arrangement of steps without exceeding the scope of the invention. The invention's scope is of course defined in the language in which the appended claims are expressed.

We claim:

1. A medical device, comprising:
an elongate shaft having a longitudinal axis and a distal end;
at least one electromagnetic coil disposed adjacent the distal end; and
a distal tip including a plurality of magnetized bulbous elements disposed about the longitudinal axis;
wherein the plurality of magnetized bulbous elements is operatively associated with the at least one electromagnetic coil.

2. The medical device of claim 1, wherein the plurality of magnetized bulbous elements is configured to translate relative to the longitudinal axis in response to activation of the at least one electromagnetic coil.

3. The medical device of claim 2, wherein the plurality of magnetized bulbous elements is configured to translate axially relative to the longitudinal axis in response to activation of the at least one electromagnetic coil.

4. The medical device of claim 2, wherein the plurality of magnetized bulbous elements is configured to translate laterally relative to the longitudinal axis in response to activation of the at least one electromagnetic coil.

5. The medical device of claim 2, wherein the plurality of magnetized bulbous elements is configured to translate rotationally relative to the longitudinal axis in response to activation of the at least one electromagnetic coil.

6. The medical device of claim 1, wherein the plurality of magnetized bulbous elements is slidingly disposed about the elongate shaft.

7. The medical device of claim 6, wherein the plurality of magnetized bulbous elements includes at least one mechanical stop associated with each magnetized bulbous element.

8. The medical device of claim 1, wherein the plurality of magnetized bulbous elements is integrally formed with the distal tip and the elongate shaft.

9. The medical device of claim 1, wherein the at least one electromagnetic coil is embedded within a wall of the elongate shaft.

10. The medical device of claim 1, wherein the at least one electromagnetic coil is disposed on an exterior surface of the elongate shaft.

11. The medical device of claim 1, wherein the at least one electromagnetic coil surrounds the elongate shaft.

12. The medical device of claim 1, wherein the at least one electromagnetic coil is disposed within a lumen of the elongate shaft.

13. The medical device of claim 1, wherein the at least one electromagnetic coil includes at least one magnet disposed within the at least one electromagnetic coil.

14. The medical device of claim 1, wherein the at least one electromagnetic coil includes a first electromagnetic coil, a second electromagnetic coil, and a third electromagnetic coil.

15. The medical device of claim 14, wherein the plurality of magnetized bulbous elements includes a first magnetized bulbous element operatively associated with the first electromagnetic coil, a second magnetized bulbous element operatively associated with the second electromagnetic coil, and a third magnetized bulbous element operatively associated with the third electromagnetic coil.

16. The medical device of claim 2, wherein the distal tip is directionally steerable using selective activation of the at least one electromagnetic coil.

17. The medical device of claim 6, wherein the plurality of magnetized bulbous elements each include a lumen extending therethrough, wherein the lumen includes a lubricious coating.

18. The medical device of claim 17, wherein at least one lumen is disposed concentric to a central axis of its respective magnetized bulbous element.

19. The medical device of claim 17, wherein at least one lumen is offset from a central axis of its respective magnetized bulbous element.

20. The medical device of claim 1, wherein the plurality of magnetized bulbous elements each include an abrasive coating disposed on an exterior surface thereof.
